SEQUENCE LISTING (13 sequences)

SEQ ID NO: 1 - DNA, 18 bp, Artificial Sequence
  Oligo 1 for PCR of murine HES-1 promotor
ctcaggcgcgcgccattg

SEQ ID NO: 2 - DNA, 20 bp, Artificial Sequence
  Oligo 2 for PCR of murine HES-1 promotor
agaggtagacaggggattcc

SEQ ID NO: 3 - protein, 10 aa, Artificial Sequence
  myc tag
EQKLISEEDL

SEQ ID NO: 4 - DNA, 20 bp, Artificial Sequence
  Oligo 1 for PCR of APP
aaccaccgtggagctccttc

SEQ ID NO: 5 - DNA, 73 bp, Artificial Sequence
  Oligo 2 for PCR of APP
ccaagcttctacaagtcctcttcagaaatcagcttttgctcgttaacgatatcgtcaacctccaccacaccat

SEQ ID NO: 6 - DNA, 28 bp, Artificial Sequence
  Oligo 1 for PCR of Notch-1
cacccgggttccctgagggtttcaaagt

SEQ ID NO: 7 - DNA, 18 bp, Artificial Sequence
  Oligo 2 for PCR of Notch-1
ccgcacgatatcgtggtg

SEQ ID NO: 8 - DNA, 26 bp, Artificial Sequence
  Oligo 3 for PCR of Notch-1
gcgttaacatctgcctgactgggctc

SEQ ID NO: 9 - DNA, 29 bp, Artificial Sequence
  Oligo 4 for PCR of Notch-1
cagttaacggtggtgggcgggctggagat

SEQ ID NO: 10 - DNA, 38 bp, Artificial Sequence
  Oligo for mutagenesis
ggagatctctgaagtgaatctggatgcagaattccgac

SEQ ID NO: 11 - DNA, 77 bp, Artificial Sequence
  Oligo 1 for PCR of NIC construct
aggatccatggtgctgctgtcccgcaagcgccggcggcagcatggccagctctggttccctgagggtttcaaagtgt

SEQ ID NO: 12 - DNA, 26 bp, Artificial Sequence
  Oligo 2 for PCR of NIC construct
gcgttaacatctgcctgactgggctc

SEQ ID NO: 13 - DNA, 6868 bp, Artificial Sequence
  Fusion between intracellular domain of murine Notch and ecto- and transmembrane domains of human APP
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcaattcccgccagggtggcgatgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgatgggttccctgagggtttcaaagtgtcagaggccagcaagaagaagcggagagagcccctcggcgaggactcagtcggcctcaagcccctgaagaatgcctcagatggtgctctgatggacgacaatcagaacgagtggggagacgaagacctggagaccaagaagttccggtttgaggagccagtagttctccctgacctgagtgatcagactgaccacaggcagtggacccagcagcacctggacgctgctgacctgcgcatgtctgccatggccccaacaccgcctcagggggaggtggatgctgactgcatggatgtcaatgttcgaggaccagatggcttcacacccctcatgattgcctcctgcagtggagggggccttgagacaggcaacagtgaagaagaagaagatgcacctgctgtcatctctgacttcatctaccagggcgccagcttgcacaaccagacagaccgcaccggggagaccgccttgcacttggctgcccgatactctcgttcagatcgtcgaaagcgccttgaggccagtgcagatgccaacatccaggacaacatgggccgtactccgttacatgcagcagtttctgcagatgctcagggtgtcttccagatcctgctccggaacagggccacagatctggatgcccgaatgcatgatggcacaactccactgatcctggctgcgcgcctggccgtggagggcatgctggaggacctcatcaactcacatgctgacgtcaatgccgtggatgacctaggcaagtcggctttgcattgggcggccgcggtgaacaatgtggatgctgctgttgtgctcctgaagaacggagccaacaaggacatcgagaacaacaaggaggagacttccctgttcctgtcgatccgccgtgagagctatgagactgccaaagtgttgctggaccactttgccaaccgggacatcacggatcacatggaccgattgccgcgggacatcgcacaggagcgtatgcaccacgatatcgtgcggcttttggatgagtacaacctggtgcggtccccacagctgcatggcactgccctgggtggcacacccactctgtctcccacactctgctcgccaaatggctaccctggcaatctcaagtctgccacacagggcaagaaggcccgcaagccaagcaccaaagggctggcttgtggtagcaaggaagctaaggacctcaaggcacggaggaagagttcccaggatggcaagggctggctgttggacagctcgtcgagcatgctgtcgcctgtggactccctcgagtcaccccatggctacttgtcagatgtggcctcgcaccccctcctcccctccccattccagcagtctccatccatgcctctcagccacctgcctggtatgcctgacacccacctgggcatcagccacttgaatgtggcagccaagcctgagatggcagcactggctggaggtagccggttggcctttgagcaccccccgccacgcctctcccacctgcctgtagcctccagtgcctgcacagtgctgagtaccaatggcaccggggctatgaatttcaccgtgggtgcaccggcaagcttgaatggccagtgtgagtggcttccccggctccagaatggcatggtgcccagccagtacaacccactacggccgggtgtgacgccgggcacactgagcacacaggcagctgggctccagcatagcatgatggggccactacacagcagcctctccaccaataccttgtccccgattatttaccagggcctgcccaacacacggctggcaacacagcctcacctggtgcagacccagcaggtgcagccacagaacttaccgctccagccacagaacctgcagccaccatcacagccacacctcagtgtgagctcggcagccaatgggcacctggggcggagcttcttgagtggggagcccagtcaggcagatgttaacgagcaaaagctgatttctgaagaggacttgtagaagcttctagatcttattaaagcagaacttgtttattgcagcttataatggttacaaataaagcaatagcatcacaaatttcacaaataaagcatttttttcactgcattctagttgtggtttgtccaaactcatcaatgtatcttatcatgtctggtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

(19) United States
(12) Patent Application Publication
(10) Pub. No.: US 2003/0059938 A1
(43) Pub. Date: Mar. 27, 2003

(54) Presenilin-deficient multipotent cell lines and screening methods for intramembrane-regulated proteolytic activities using these lines

(76) Inventors: Wim Annaert, Kontich (BE); Bart De Strooper, Hoeilaart (BE); An Herreman, Lier (BE); Luc Schoonjans, Wilsele (BE); Lutgarde Serneels, Itegem (BE)

Correspondence Address: TRASK BRITT, P.O. BOX 2550, SALT LAKE CITY, UT, 84110, US

(21) Appl. No.: 10/228,531

(22) Filed: Aug. 26, 2002

Related U.S. Application Data
(63) Continuation of application No. PCT/EP01/02127, filed on Feb. 21, 2001.

(30) Foreign Application Priority Data
Feb. 25, 2000 (EP) 00200671.6

(51) Int. Cl. C12N 5/06; C12N 5/08

(52) U.S. Cl. 435/354; 435/368

(57) ABSTRACT

The present invention relates to the field of neurological and physiological dysfunctions associated with Alzheimer's disease, more particularly to mutant embryonic stem (ES) cell lines characterized by no detectable γ-secretase activity, derived from double presenilin (PS 1 and PS 2) knockout mice embryos. These cell lines can be used for in vitro screening of molecules and products involved in regulated intramembrane proteolysis of proteins such as the PP, the APP-like proteins, Notch, Ire-1p, and other integral membrane proteins to identify proteases responsible for the latter proteolysis, like gamma-secretases, or proteins involved in the control of these proteolytic activities. These mutant ES cell lines can be manipulated to differentiate into fibroblasts, neurons, or myocytes or can be used to generate novel transgenic mice. Moreover, a reporter system comprising a chimeric molecule to detect the above-mentioned intramembrane proteolysis or modulators thereof has been developed.

### CROSS-REFERENCE TO RELATED APPLICATION

This application is a continuation of International Application Number PCT/EP01/02127 filed on Feb. 21, 2001 designating the United States of America, International Publication No. WO 01/62897 (Aug. 30, 2001), published in English, the contents of the entirety of which are incorporated by this reference.

### TECHNICAL FIELD

The present invention relates to the field of neurological and physiological dysfunctions associated with Alzheimer's disease. Particularly, the invention relates to novel, mutant embryonic stem (ES) cell lines characterized by no detectable γ-secretase activity. More particularly, the invention relates to novel, mutant embryonic stem (ES) cell lines derived from double presenilin (presenilin 1 and presenilin 2) knockout mice embryos.

### BACKGROUND

Alzheimer's disease (AD) is a neurological disorder that is clinically characterized by the progressive loss of intellectual capabilities, most prominently memory, but later also by disorientation, impairment of judgment and reasoning, and ultimately full dementia. The patients finally fall into a severely debilitated, immobile state between 4 and 12 years after onset. Worldwide, about 20 million people suffer from Alzheimer's disease (AD). A small fraction of AD cases are caused by autosomal dominant mutations in the genes encoding presenilin (PS) proteins 1 and 2 and the amyloid-β precursor protein (APP). It has been shown that mutations in APP, PS1 and PS2 alter APP metabolism such that more of the insoluble, pathogenic Aβ peptide (Aβ42) is produced. This Aβ42-peptide forms amyloid fibrils more readily than the Aβ40-peptide, which is normally produced via wild-type APP, PS1 and PS2. These insoluble amyloid fibrils are deposited in amyloid plaques, one of the neuropathological hallmarks in the brains of patients suffering from AD. These Aβ-peptides are generated from the amyloid precursor protein (APP) by distinct proteolytic activities. The γ-secretase was recently identified and is a type I integral membrane aspartyl protease, also called BACE (Vassar et al., 1999, Science 286, 735). BACE cleaves APP at the aminoterminus of the amyloid peptide sequence in APP. The elusive γ-secretase cuts APP at the carboxyterminus of the amyloid peptide. Although it is still unclear whether one, two or several different enzymes are involved in this process, it can be stated that the secretases are important and are molecular targets for drug discovery since it is believed that abnormal processing of APP is involved in the pathogenesis of both genetic and sporadic Alzheimer's disease. It is also clear that the molecular identification of endogenous proteins involved either directly or indirectly in secretase activities is of uttermost importance.

Recent research has demonstrated the involvement of presenilins in the formation of amyloid-β through their effects on γ-secretase(s). These findings establish a genetic link between presenilins and γ-secretase(s) and make them potential molecular targets for developing compounds to prevent or treat AD.

Presenilins (PS) are polytransmembrane proteins located in the endoplasmic reticulum and the early Golgi apparatus. Missense mutations cause familiar Alzheimer's disease (AD) in a dominant fashion. The exact pathogenic mechanism underlying the disease process is not fully unraveled, but it is fairly established that most PS missence mutations effect the processing of the amyloid precursor protein (APP), resulting in an increased generation of the longer form of the amyloid peptide (the Aβ42-peptide), which is a major component of the amyloid plaques in patients as stated above.

The inactivation of the PS1-gene in mice results in a severe lethal phenotype, characterized by late embryonic lethality, disturbed somitogenesis, mid-line closure deficiencies and malformations of the central nervous system, most significantly underdevelopment of the subventricular zone and a neuronal migration disorder mimicking human lissencephaly type II (Hartmann et al., 1999, Curr. Biol. 9, 719). Cell biological studies in PS1-deficient neurons have demonstrated that PS1 deficiency interferes with the γ-secretase-mediated proteolysis of the transmembrane domain of APP and an estimated reduction of 85% γ-secretase-mediated proteolysis was observed (De Strooper et al., 1998, Nature 391, 387). However, it is unknown which additional factors modulate the activity of the γ-secretase.

Presenilin 1 appears also to be involved in the proteolytic processing of the transmembrane domain of other proteins like Notch, a signaling protein involved in cell fate decisions (De Strooper et al., 1999, Nature 398, 518), and possibly Ire1p, a protein involved in the control of the unfolded protein response (Niwa et al., 1999, Cell 99, 691). This type of proteolytic processing has been recently called “regulated intramembrane proteolysis” (rip) (Brown et al., 2000, Cell 100, 391). Recently, Wolfe and colleagues (Wolfe et al., 1999, Nature 398, 513) proposed that PS1 itself is an unusual aspartyl protease. Owing to their involvement in the cleavage of APP, the presenilins may turn out to be the long-sought-after γ-secretase. Aspartyl proteases, like the presenilins, require two aligned aspartate residues in their catalytic domain. It should be emphasized, however, that direct evidence for the conclusion that PS1 or PS2 represents the γ-secretase activity itself is still lacking and that direct evidence that presenilins have catalytic activity has not been provided. Moreover, it is unclear whether all proteolytic cleavages in which presenilin is involved can be performed by one identical protease, since the primary amino acid sequences of the different cleaved proteins is quite variable. It is, therefore, certainly possible that the presenilins influence γ-secretase activity indirectly. Presenilins may, for instance, control membrane insertion of γ-secretase or may behave as cofactors stimulating their catalytic activity.

An analogy can be drawn with the regulation of the site 1 cleavage of sterol regulatory element binding proteins, which is controlled by a multitransmembrane cleavage-activating protein located in the ER.

Recent evidence also implies the presenilins in Wnt/β-catenin signaling (Zhang et al., Nature 395:698-702,1998; Kang et al., J. Neurosci.19: 4229-4237, 1999). While their exact role in this pathway remains controversial, it is established that presenilins can interact with β-catenin.

A major problem with the hypothesis that presenilins are proteases is their subcellular localization. Presenilin proteins have been localized to early transport compartments, whereas abundant γ-secretase activity is thought to be associated with the late transport compartments and the endosomal pathway (Annaert et al., 1999, J. of Cell Biology 147, 277).

The interpretation of all studies performed until now has been complicated by the fact that at least two presenilin genes do exist. Most studies did not take into account the influence of the PS2-gene. In contrast to PS1 knockout mice, PS2 knockout mice are viable and fertile and develop only mild pulmonary fibrosis and hemorrhage with age (Herreman et al., 1999, Proc. Natl. Acad. Sci. 96, 11872). Quite surprisingly and unexpectedly, the absence of PS2 does not detectably alter the processing of amyloid precursor protein.

In a further step, the complete deletion of both PS2 and PS1 genes was therefore pursued. The phenotype of these mice closely resembles the phenotype of mice that are fully deficient in Notch-1. These observations demonstrate that PS1 and PS2 have partially overlapping functions and that PS1 is essential and PS2 is redundant for normal Notch signaling during mammalian embryological development. Biochemical analysis of the exact effects of the double PS deletion on Notch signaling, APP processing, the UPR and other biological processes is, however, hampered by the fact that only a limited number of cells can be obtained from such embryos. This has been circumvented by immortalizing these cells using transfection with large T or myc cDNA constructs. Although this allows one to obtain large amounts of presenilin negative cells, these procedures also largely interfere with important cellular signaling mechanisms and also lead to genetic instability of the cells. It is, therefore, difficult to assess correctly to what extent phenotypical alterations in these cells are caused by the presenilin deficiency in se or by secondary alterations caused by the immortalization procedure.

It is thus clear that the development of clean and genetically stable cell lines, without activity of PS1 and PS2, is needed in order to understand the above-described biological pathways, and especially the role of the presenilins, their mutations and deficiencies in the pathogenesis of AD.

### SUMMARY OF THE INVENTION

The present invention provides embryonic stem (ES) cell lines generated from double presenilin (PS1 and PS2) knockout mice. Surprisingly, given the residual γ-secretase activity in PS1 knockout cells and the absence of effects on γ-secretase activity in PS2 knockout cells, it was found that γ-secretase activity dropped to an undetectable level in these mutant cell lines. Accordingly, the latter cell lines can be used to screen for γ-secretase activity and modulators thereof. The present invention also provides a reporter system to detect γ-secretase activity and modulators thereof.

The present invention provides embryonic stem cell lines in which the residual γ-secretase activity is reduced by more than 90%, preferentially more than 99%, and more preferentially more than 99.9%, compared to the γ-secretase activity in embryonic stem cell lines derived from corresponding wild-type mice.

A particular ES cell line of the present invention has been deposited with the Belgian Coordinated Collections of Microorganisms (BCCMTM), Laboratorium voor Moleculaire Biologie—Plasmidencollectie (LMBP), Universiteit Gent, K. L. Ledeganckstraat 35, B-9000 Gent, Belgium, and has been given accession number: LMBP 5472CB.

The present invention thus provides a clean mutant mammalian environment which allows one to exclude the contribution of endogenously expressed presenilins to any induced γ-secretase activity or other proteolytic activities. Furthermore, such cell lines provide a perfect background to investigate the biochemical effects of transfected presenilins containing mutations, since no interference from endogenously wild-type presenilins is possible.

The novel cell lines can be used for in vitro screening of molecules and products involved in regulated intramembrane proteolysis of proteins such as the amyloid precursor protein, the amyloid precursor like proteins, Notch, Ire-1p, and possibly other integral membrane proteins. These cell lines can further be used to identify proteases responsible for the latter proteolysis, in particular for identifying γ-secretases, or for the identification of proteins involved in the control of these proteolytic activities. In addition, these mutant ES cell lines can be manipulated to differentiate into several specialized cell lines such as fibroblast, neurons, myocytes and other differentiated cell lines or can be used to generate novel transgenic mice. Moreover, the present invention discloses a reporter system comprising a chimeric molecule to detect the above mentioned intramembrane proteolysis or modulators thereof.

In a preferred embodiment, the double presenilin mutant ES cell line can be used as a tool to isolate and identify γ-secretase candidates and genetic modulators of γ-secretase activity. By isolation it is meant that standard molecular biology tools such as complementation, screening or selection cloning methods with a genomic or cDNA library are used to transfect the cells and to induce γ-secretase activity. It is clear that also recombinant virus libraries, such as adenoviral, lentiviral or retroviral libraries, can be used.

In another embodiment, the double presenilin mutant ES cell line can be used to generate membranes or protein extracts that can be complemented biochemically with fragments of presenilin or other proteins to reconstitute gamma-secretase activity in vitro (Li et al., Proc. Natl. Acad. Sci. 97; 6138-43, 2000). The aforementioned methodology are only examples and do not rule out other possible approaches of using these cells that could lead to potential γ-secretase candidates. Since presenilins are required for the maintenance of γ-secretase activity in normal conditions, it is anticipated that screening assays will yield parts of γ-secretase that are devoid of putative regulatory domains that interact with presenilins. Such partial clones can then be used to obtain the complete cDNA of γ-secretase and/or other proteases involved in regulated intramembrane proteolysis (see above), more in particular of APP.

Restoration of proteolytic activity can be followed by different means, to give only a few examples: ELISA assays or other assays measuring amyloid peptide production, or assays measuring Notch cleavage using luciferase reporter systems, or other assays. To increase the sensitivity of such assays, it can be considered to stably transfect the ES cells with cDNA's encoding APP (human, containing clinical or synthetic mutations), Notch (possibly mutated or modified), or other proteins and reporters useful for such assays.

In another embodiment of the invention, the double mutant presenilin ES cell line can be used as a cellular background to express presenilin clinical mutations. Such a cell line can then be used to screen for inhibitors that specifically inhibit the production of pathogenic amyloid-β42-peptide. Indeed, the double mutant presenilin ES cell line transformed with an Alzheimer's disease-causing PS1 mutation is predicted to produce predominantly the amyloid-β42 form whereas the mutant ES cell line transformed with the wild-type PS1 is predicted to produce mostly the nonpathogenic amyloid-β40 peptide. These cell lines can thus be used in differential drug screening approaches to identify compounds which inhibit preferentially the amyloid-β42 formation and not the amyloid-β40 peptide generation. By comparing the differential effect of compounds on the amyloid peptide production in the mutant cell lines overexpressing wild-type presenilin and clinical mutant presenilin, compounds affecting the pathological amyloid peptide (amyloid-β42 peptide) production can specifically be detected. A compound able to interfere with the formation of amyloid-β42 peptide and not with the formation of amyloid-β40 peptide should at least have a 20% reduction in amyloid-β42 peptide formation, preferentially at least a 50% reduction in amyloid-β42 peptide formation and more preferentially at least a 90% reduction in amyloid-β42 peptide formation.

In another embodiment, the invention provides a method for the production of a pharmaceutical composition comprising the usage of an embryonic stem cell line to identify a gene coding for a protein having gamma-secretase activity or a compound that specifically interferes with the formation of the Aβ42-peptide and not with the formation of the Aβ40-peptide, and, furthermore, mixing the gene or compound identified or a derivative or homologue thereof with a pharmaceutically acceptable carrier. The administration of a gene or compound or a pharmaceutically acceptable salt thereof may be by way of oral, inhaled or parenteral administration. The active compound may be administered alone or preferably formulated as a pharmaceutical composition. A unit dose will normally contain 0.01 to 50 mg, for example, 0.01 to 10 mg, or 0.05 to 2 mg of the compound or a pharmaceutically acceptable salt thereof. Unit doses will normally be administered once or more than once a day, for example, 2, 3, or 4 times a day, more usually 1 to 3 times a day, such that the total daily dose is normally in the range of 0.0001 to 1 mg/kg; thus a suitable total daily dose for a 70 kg adult is 0.01 to 50 mg, for example 0.01 to 10 mg or more usually 0.05 to 10 mg. It is greatly preferred that the compound or a pharmaceutically acceptable salt thereof is administered in the form of a unit dose composition, such as a unit dose oral, parenteral, or inhaled composition. Such compositions are prepared by admixture and are suitably adapted for oral, inhaled or parenteral administration, and as such may be in the form of tablets, capsules, oral liquid preparations, powders, granules, lozenges, reconstitutable powders, injectable and infusable solutions or suspensions or suppositories or aerosols. Tablets and capsules for oral administration are usually presented in a unit dose and contain conventional excipients such as binding agents, fillers, diluents, tableting agents, lubricants, disintegrants, colorants, flavorings, and wetting agents. The tablets may be coated according to well-known methods in the art. Suitable fillers for use include cellulose, mannitol, lactose and other similar agents.

Suitable disintegrants include starch, polyvinylpyrrolidone and starch derivatives such as sodium starch glycollate. Suitable lubricants include, for example, magnesium stearate. Suitable pharmaceutically acceptable wetting agents include sodium lauryl sulphate. These solid oral compositions may be prepared by conventional methods of blending, filling, tableting or the like. Repeated blending operations may be used to distribute the active agent throughout those compositions employing large quantities of fillers. Such operations are, of course, conventional in the art. Oral liquid preparations may be in the form of, for example, aqueous or oily suspensions, solutions, emulsions, syrups, or elixirs, or may be presented as a dry product for reconstitution with water or other suitable vehicle before use. Such liquid preparations may contain conventional additives such as suspending agents, for example, sorbitol, syrup, methyl cellulose, gelatin, hydroxyethylcellulose, carboxymethyl cellulose, aluminum stearate gel or hydrogenated edible fats, emulsifying agents, for example, lecithin, sorbitan monooleate, or acacia; non-aqueous vehicles (which may include edible oils), for example, almond oil, fractionated coconut oil, oily esters such as esters of glycerine, propylene glycol, or ethyl alcohol; preservatives, for example, methyl or propyl p-hydroxybenzoate or sorbic acid, and if desired conventional flavoring or coloring agents. Oral formulations also include conventional sustained-release formulations, such as tablets or granules having an enteric coating. Preferably, compositions for inhalation are presented for administration to the respiratory tract as a snuff or an aerosol or solution for a nebulizer, or as a microfine powder for insufflation, alone or in combination with an inert carrier such as lactose. In such a case, the particles of active compound suitably have diameters of less than 50 microns, preferably less than 10 microns, for example, between 1 and 5 microns, such as between 2 and 5 microns. A favored inhaled dose will be in the range of 0.05 to 2 mg, for example, 0.05 to 0.5 mg, 0.1 to 1 mg or 0.5 to 2 mg. For parenteral administration, fluid unit dose forms are prepared containing a compound of the present invention and a sterile vehicle. The active compound, depending on the vehicle and the concentration, can be either suspended or dissolved. Parenteral solutions are normally prepared by dissolving the compound in a vehicle and filter sterilizing before filling into a suitable vial or ampule and sealing.

Advantageously, adjuvants such as a local anaesthetic, preservatives and buffering agents are also dissolved in the vehicle. To enhance the stability, the composition can be frozen after filling into the vial and the water removed under vacuum. Parenteral suspensions are prepared in substantially the same manner except that the compound is suspended in the vehicle instead of being dissolved and sterilized by exposure to ethylene oxide before suspending in the sterile vehicle. Advantageously, a surfactant or wetting agent is included in the composition to facilitate uniform distribution of the active compound. Where appropriate, small amounts of bronchodilators, for example, sympathomimetic amines such as isoprenaline, isoetharine, salbutamol, phenylephrine and ephedrine; xanthine derivatives such as theophylline and aminophylline and corticosteroids such as prednisolone and adrenal stimulants such as ACTH may be included. As is common practice, the compositions will usually be accompanied by written or printed directions for use in the medical treatment concerned.

Another embodiment involves the differentiation of the obtained ES cell lines towards neurons and in particular towards post-mitotic neurons.

In another embodiment, the double mutant presenilin ES cell line can be used to differentiate into many cell lineages, including heart muscle cells, blood islands, pigmented cells, macrophages, epithelia, and fat-producing adipocytes.

In another embodiment, the double mutant presenilin ES cell line can be used to clarify the role presenilins play in the unfolded protein response (UPR-response).

In another embodiment, the double mutant presenilin ES cell line can be transformed with a specific presenilin mutant, such as a pathogenic presenilin Alzheimer's disease-causing gene. The resulting transformed mutant ES cell line with the specific presenilin mutant can be injected back into a recipient blastocyst that is then carried to term in a female host. In this way, it is possible to generate very specific, clean transgenic mice in which there is no interference by the wild-type presenilin homologous genes, neither of the other presenilin genes.

In another embodiment, the double mutant presenilin ES cell line can be used to unravel the role of Notch in differentiation or to unravel the Notch signaling pathway. As a preferred but not limited example, the double mutant presenilin ES cell line can be transformed with an inducible PS1 gene, and in a gene expression profiling experiment, the gene expression can be monitored before and after induced expression of PS1.

In another embodiment, the double mutant presenilin ES cell line can be used to unravel the presumed role PS2 is playing in inducing apoptosis.

Another embodiment is the use of these mutant ES cell lines in a cell-free assay whereby vesicle budding from ER membranes is studied. The profiling of the protein composition of the generated transport vesicles can then be investigated by two-dimensional electrophoresis and amino acid sequencing of protein spots that are either increased or decreased in comparison with spots present in the profile of similar gel profiles obtained from wild-type cells.

Another aspect of the invention relates to the realization, especially in the field of cell signaling, that certain transmembrane proteins can be cleaved within the transmembrane domain to liberate cytosolic fragments that enter the nucleus to control gene transcription. This mechanism, called regulated intramembrane proteolysis (Rip), influences processes as diverse as cellular differentiation, lipid metabolism, and the response to unfolded proteins (Brown et al., 2000, Cell 100, 391). Currently five animal proteins are known or postulated to undergo Rip: SREBP, APP, Notch, Ire1 and ATF6 (Brown et al., 2000, Cell 100, 391). In some of these examples, the information is still fragmentary, and in no case do we have a complete picture of the processing events. Furthermore, there are no reporter systems described that can detect Rip and also the processing enzymes (e.g., proteases) have not been identified with certainty. The present invention provides a system to detect Rip and to identify processing enzymes (e.g., proteases) and modulators of the processing enzymes.

Thus, a reporter system was generated that can be used to detect intramembrane proteolytic processing. The reporter system comprises a chimeric molecule further comprising a fusion between a transcription factor and a transmembrane domain that is known to be a substrate for proteolytic processing, and a reporter construct that can detect the proteolytic processing of the chimeric transcription factor. When intramembrane cleavage occurs, the chimeric transcription factor is not kept sequestered anymore in the membrane and can shuttle to the nucleus where it activates the expression of a reporter gene. The reporter gene is kept under control of an inducible promoter of which activity is dependent on the release of the specific, membrane-sequestered transcription factor.

In a further embodiment, the chimeric molecule that is used in the reporter system comprises a fusion between the intracellular domain of Notch and the transmembrane domain of APP. The reporter system can be used to detect intramembrane proteolytic processing by γ-secretase. The transmembrane domain of APP comprises the γ-secretase cleavage site.

In yet another embodiment, the chimeric molecule of the reporter system comprises a fusion between the intracellular domain of Notch and the ecto- and transmembrane domains of APP and is set forth by SEQ ID NO: 13. In SEQ ID NO: 13, APP starts at nucleotide 275 and ends at 2266. The transmembrane domain of APP starts at 2147 and ends at 2208. The intracellular domain of Notch starts at 2273 and ends at 4249. The myc tag starts at 4256 and ends at 4285. The stop codon of the chimeric protein is between 4286-4288.

In yet another embodiment, the reporter system of the present invention can be used to screen for modulators and/or proteases for intramembrane proteolytic processing. In a particular embodiment, the genetic background that is used to screen for the modulators and/or proteases is devoid of the activity of intramembrane proteolytic processing. In such a genetic background, there is no background activation of the reporter construct since the chimeric transcription factor is maintained in the membrane. For example, the double presenilin ES cell line of the present invention is a perfect tool for the isolation of the γ-secretase or modulators of γ-secretase activity. By “isolation,” it is preferentially meant “screening,” and more preferentially “selection.” As has been said before, γ-secretase is an enzyme involved in Rip of the transmembrane domain of APP. In order to establish a successful cloning experiment, the mutant ES cell line is first adapted into a suitable reporter ES cell line with the introduction, by transfection, of the above-described reporter system. Screening for γ-secretase (a protease) or modulators of γ-secretase activity can be carried out with a suitable reporter gene, such as the green fluorescent protein. However, one can also select for γ-secretase or modulators of γ-secretase activity by use of a selection marker, such as the neomycin phosphotransferase gene, under control of an inducible promoter, which is dependent on the release of the specific, sequestered transcription factor. As such, the transcription factor is released from its membrane localization and, consequently, provides antibiotic resistance after cleavage by the γ-secretase or γ-secretase modulators. Selection or screening for the γ-secretase activity is carried out by the transfection of a genetic library to the reporter ES cell line. Preferentially, this library is a mammalian genomic library, and more preferentially the library is a cDNA library under control of a suitable promoter, and even more preferentially the cDNA library is of neuronal origin. It is clear that recombinant virus libraries, such as adenoviral, lentiviral or retroviral libraries, can also be used. The aforementioned libraries are only examples and do not rule out other possible cloning approaches that could lead to the identification of potential γ-secretase candidates or genetic modulators of γ-secretase. By modulators, here it is meant compounds or genes that influence the activity of the proteases sought, as such a modulator can enhance (activator) or diminish (suppressor) the activity. For instance, but not limited to, a modulator of γ-secretase can be a protein binding to γ-secretase or a protein that forms part of a multi-protein complex that has γ-secretase activity.

In another embodiment, the reporter system of the present invention is used to screen for modulators and/or proteases in a cell line with a wild-type genetic background, wherein the protease(s) that provoke the intramembrane proteolytic processing are present. In such a cell line there is usually a constitutive expression level of the reporter construct. As such, a selection or screening system can be set up by comparing the expression level of the reporter construct in the cell line with the same but transfected or compound-treated cell line. As such, in a screening or selection experiment, a higher level of the protease, such as, for example, the γ-secretase or a modulator thereof, in the cell line (e.g., by transfection with at least one gene), can be detected by a higher activation of the reporter construct with respect to the nontransfected cell line. Conversely, a lower activity of the γ-secretase can also be detected by a specific compound or a modulator when compared with the nontransfected or noncompound-treated cell line. Finally, in another embodiment, a process is described to construct a reporter system. See FIGS. 4C and 4D. The process comprises the formation of two specific constructs. The first construct is made by splicing the sequence of a transcription factor to a transmembrane domain that is known to be a substrate for proteolytic processing and the second construct is made by splicing of a genetic element that is responsive to the transcription factor to a reporter gene.

### BRIEF DESCRIPTION OF THE DRAWINGS

In the drawings, which illustrate what is currently considered to be the best mode for carrying out the invention:

FIG. 1 depicts a southern blot analysis of ES clones obtained from individual blastocysts. ES cells were harvested and genomic DNA was isolated. After restriction with Kpn1, Southern blotting was performed using the 5′ PS1 probe (Saftig et al., Molecular biology of Alzheimer's Disease, Harwood Academic Publishers, editor Christian Haass, pp 235-246). A 6.8 kb band and 4.5 kb band are obtained, indicating respectively a wild-type or a targeted PS1 allele. As one can observe, lanes a, b, f, g and i are double presenilin mutant ES cells.

FIG. 2 depicts lower βA4 production in double PS1 and PS2 knockout ES cells. ES cells were transduced with recombinant Semliki Forest Virus to induce expression of APP/Sw (De Strooper et al., 1995, EMBO J. 14, 4932). After transduction, cells were metabolically labeled with ³⁵S-methionine (500 μCi/ml) and, after 4 hours, the conditioned medium was collected and cells were lysed. The conditioned medium was used in an immunopreciptation reaction using amyloid peptide-specific antibodies B7/7 (De Strooper et al., 1998, Nature 391, 387). No amyloid peptide production is observed in the double deficient ES cells.

FIG. 3 shows accumulation of APP carboxyterminal fragments. ES cells were transduced (APPSw) or not transduced (Co) with recombinant Semliki Forest Virus to induce expression of APP/Sw. Cells were labeled as in FIG. B and cell extracts were generated as described (De Strooper et al., 1995, EMBO J. 14, 4932). APP antibodies B11/4 recognizing the carboxyterminal end of APP were used to immunoprecipitate APP (holo-APP) and carboxyterminal fragments of APP (α-stubs and β-stubs). An accumulation of APP carboxyterminal fragments is observed in the first lane, indicating inhibition of the normal turn over of these fragments in the double knockout cells (De Strooper et al., 1998, Nature 391, 387).

FIG. 4A shows cell-based γ-secretase assay. The APP ectodomain and transmembrane domain are fused to the intracellular domain of Notch. Luciferase cDNA is fused to the Hes-1 promotor fragment as indicated in the text. Cells are transfected with these constructs. Proteolytic release of the Notch intracellular domain by a γ-secretase activity induces luciferase activity as indicated, allowing one to monitor efficiently γ-secretase activity. (CSL is present in the transfected cells.)

In FIG. 4B, different constructs are displayed. The first series are chimeric proteins containing progressively shortened Notch intracellular domain fragments fused to wild-type APP (see text for details). The next construct contains the Swedish mutation of APP to increase β-secretase cleavage. The last construct is similar to the first construct, but the APP ectodomain was truncated at the β-secretase site.

FIG. 4C depicts how pSG5APP-NIC and pSG5APPsw-NIC constructs were transfected in Cos and Hela cells. Reaction with antibodies against the N terminus of APP (22C11), or against the cytoplasmic domain of APP or with the monoclonal 9E10 (myc tag) demonstrated in western blotting a protein with a molecular mass around 150 kDa corresponding to the predicted fusion protein (indicated as APP:NIC). α or β secretase cleaves the extracellular domain of APP, producing a soluble ectodomain APP and a membrane-associated carboxyterminal fragment (APP:NIC CT fragment). Amyloid-β peptide and p3 fragment are also produced, as indicated in the final panel. The production of amyloid and p3 peptide is less efficient from the chimeric construct than from wild-type APP. One possible reason is that the endocytosis signals in the APP cytoplasmic tail, important for amyloid production in wild-type APP, are not present in the chimeric protein.

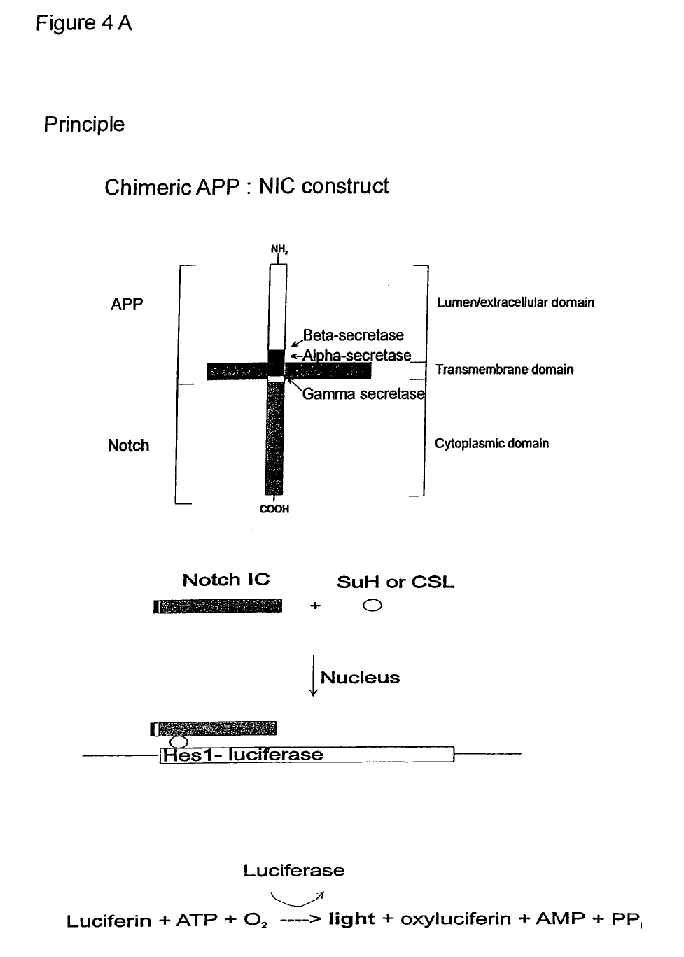

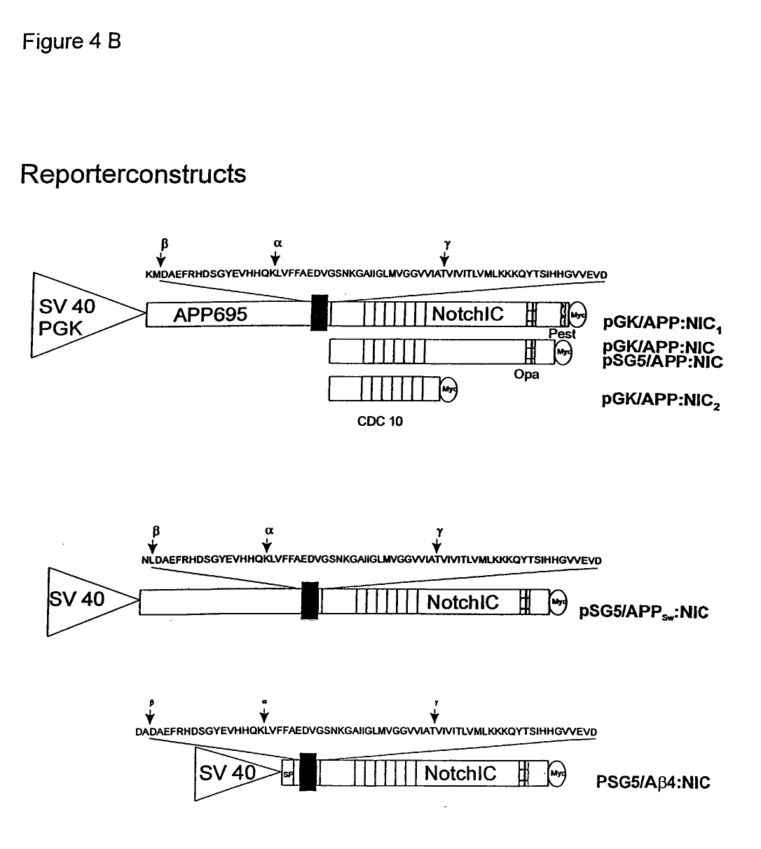

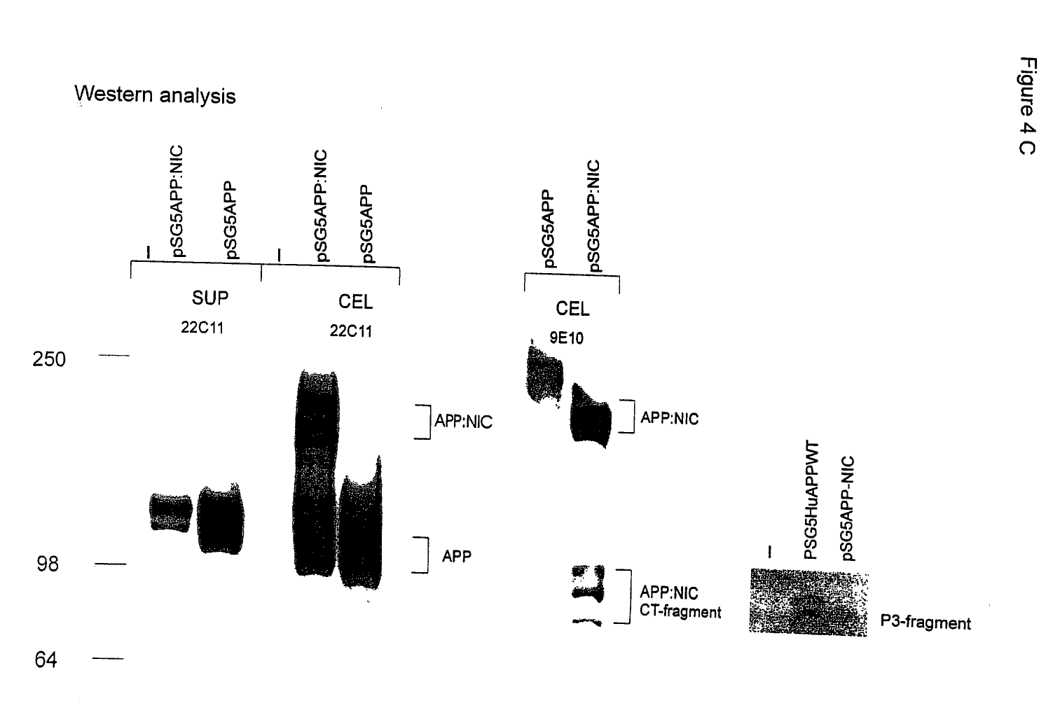

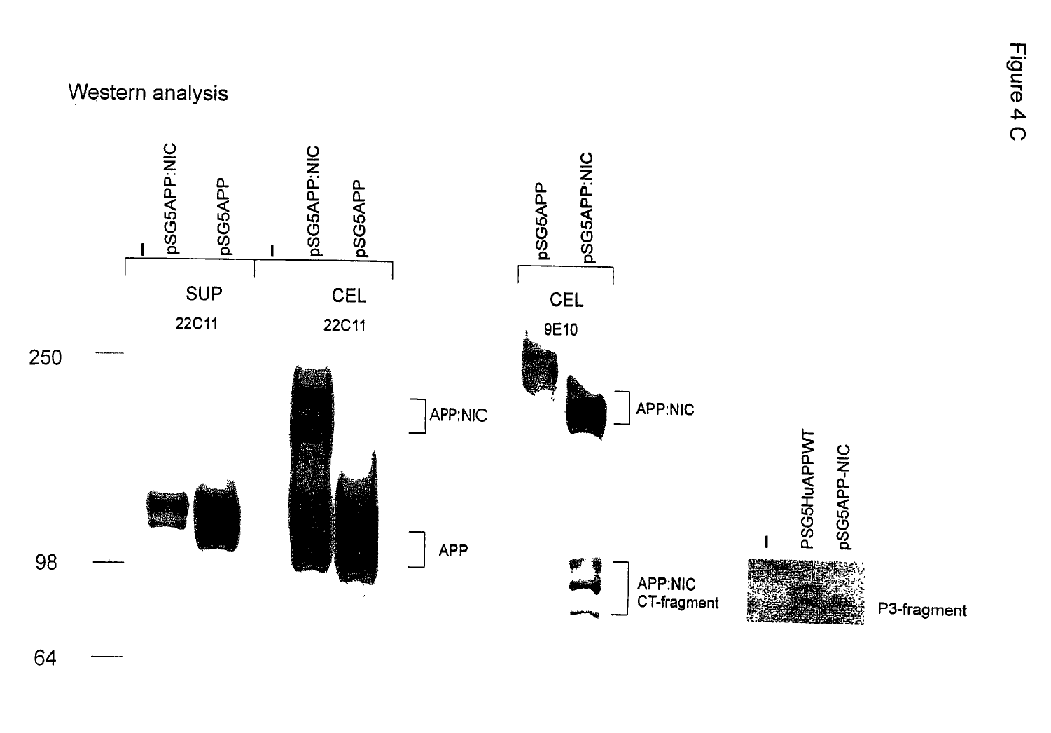

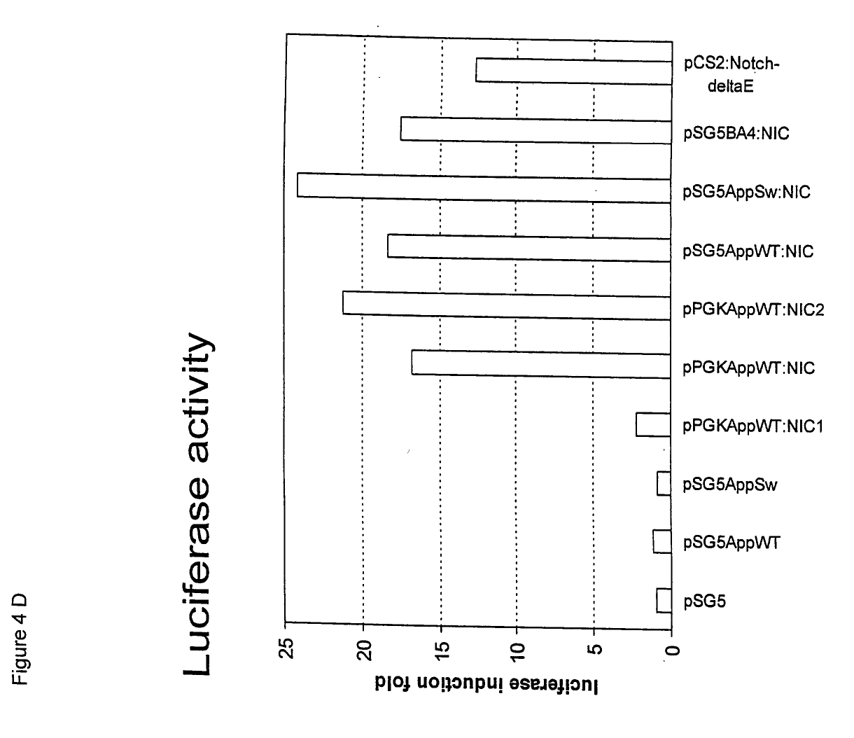

In FIG. 4D, the results with different constructs are displayed. Significant induction of luciferase activity is shown after transfection with the chimeric constructs. The different APP:NIC chimeric proteins have very similar induction efficiencies.

### DETAILED DESCRIPTION OF THE INVENTION

The following paragraphs clarify some terms used above and in the claims:

The “ES cell” is preferably a culture cell established from the inner cell mass of a murine embryo, usually isolated at the age of 3.5 days.

“Compound” means any chemical or biological compound, including simple or complex inorganic or organic molecules, peptides, peptido-mimetics, proteins, antibodies, carbohydrates, nucleic acids or derivatives thereof.

“Wild-type” is an animal (e.g., a mouse) or cell line that is isogenic with the mutant animal (e.g., mouse) or cell line except for the mutation or mutations induced in the mutant.

“Gene” means a functional promoter sequence fused to a sequence that can be transcribed, due to the activity of the promoter, into mRNA, and subsequently translated into protein, eventually after processing the mRNA by a process such as mRNA splicing. The promoter may be the endogenous promoter of the transcribed sequence, or a heterologous promoter.

“Mutant (ES) cell line” is an (ES) cell line genetically modified by a procedure known to people skilled in the art, such as random mutagenesis, retroviral or adenoviral or lentiviral insertion, transposon mutagenesis, heterologous or homologous recombination.

A “mutant gene” is a variant of the wild-type gene that differs from the wild-type gene by a change, insertion and/or deletion of at least one amino acid.

“Transgenic mouse” is a mouse derived from the mutant ES cell line. It also includes an individual animal in all stages of development, including embryonic and fetal stages.

A “transgenic animal” is an animal containing one or more cells bearing genetic information received, directly or indirectly, by deliberate genetic manipulation at a subcellular level, such as by microinjection or infection with recombinant virus. This introduced DNA molecule may be integrated within a chromosome, or it may be extra-chromosomally replicating DNA.

The term “germ cell-line transgenic animal” refers to a transgenic animal in which the genetic information was introduced into a germ line cell, thereby conferring the ability to transfer the information to offspring. If such offspring in fact possess some or all of that information, then they too are transgenic animals. The information may be foreign to the species of animal to which the recipient belongs, foreign only to the particular individual recipient, or genetic information already possessed by the recipient. In the last case, the introduced gene maybe differently expressed compared to the native endogenous gene. A variant transgenic animal is a knockout animal possibly prepared according to Capecchi, 1989, Trends Genet. 5, 70.

A “reporter gene” is generally incorporated in a reporter construct. A “reporter gene” is a DNA molecule that expresses a detectable gene product, which may be RNA or protein. The detection may be accomplished by any method known to one skilled in the art. For example, detection of mRNA expression may be accomplished by using Northern blots and detection of protein may be accomplished by staining with antibodies specific to the protein. Preferred reporter genes are those that are readily detectable comprising chloramphenicol acetyl transferase, luciferase, beta-galactosidase and alkaline phosphatase. Other reporter genes are genes providing antibiotic resistance such as the neomycine phosphotransferase gene.

As used herein, “APP695” refers to the 695 amino acid residue long polypeptide encoded by the human APP gene (Kitaguchi et al. (1988) Nature 331:530).

A “chimeric molecule” means a molecule not naturally encountered in nature; in this invention, the chimeric molecule is obtained by “splicing” part of the genetic information residing from two different genes encoding proteins.

“Splicing” is a term used by molecular biologists and here means “joined” by known methods in genetic engineering (e.g., PCR-cloning, restriction enzyme mediated cloning).

A “transmembrane domain” is a protein domain that crosses a membrane (e.g., endoplasmic reticulum, plasma membrane).

The terms “transformed” and “transfected” are used interchangeably and refer to the process by which heterologous DNA or RNA is transferred or introduced into an appropriate host cell.

### EXAMPLES

I. Results

1. Generation of Double Presenilin Knockout Mice

The generation of PS1+/− and PS2−/− mice have been described previously (De Strooper et al., 1998, Nature 391, 387; Herreman et al., 1999, Proc. Natl. Acad. Sci. 96, 11872). All mice used have a C57B6/J black×129Sv genetic background.

PS2−/− mice were crossed with PS1+/− mice to obtain PS1+/−PS2+/− double heterozygous mice. Double heterozygotes were then crossed with PS2−/− homozygous mice to obtain PS1+/−PS2−/− mice. Even with only one active PS1 gene left, these mice remain completely viable and fertile. Liveborn double homozygous offspring from these heterozygous intercrosses could not be detected. At E9.5 homozygous PS1−/−PS2−/−embryos could be recovered in a nearly Mendelian distribution (15/66), but embryos were developmentally retarded by approximately half a day when compared to heterozygous littermates. Vasculogenesis of the yolk sac was delayed in most of the mutants. Although an initial vascular plexus and primitive red blood cells had formed, organization into a discrete network of vitelline vessels was always lacking. Furthermore, yolk sacs did not expand properly and often had a blistered appearance. The embryo itselfwas always devoid of blood circulation and appeared posteriorly truncated. Heart development was largely unaffected, with the exception of an occasional enlarged pericardial sac. Somitogenesis had begun and turning occurred in the majority of the mutants. The optic and otic vesicle, the first branchial arch and the forelimb buds were visible. Mutants had a vestigial fore- and hindbrain, and fusion of headfolds was delayed. The neural tube had often a kinked appearance, which may be secondary to the circulation problems. This phenotype of the double deficient embryos is clearly different from that of PS2−/− embryos, which appear normal, and PS1−/− embryos, which are only marginally retarded at E9.

2. Generation of Double Presenilin-deficient Embryonic Stem Cells

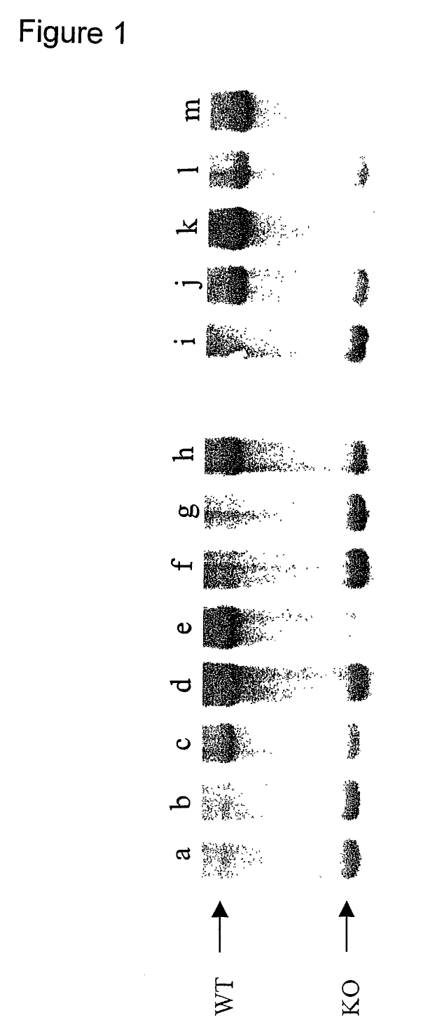

It is well known in the art that cells from the inner cell mass of rabbit, mouse, and rat blastocysts can be maintained in tissue culture under conditions where they can be propagated indefinitely as pluripotent embryonic stem (ES) cells (Thomson et al., 1998, Science 282, 1145). As such, blastocysts from double presenilin knockout mice were rescued from 2 day-old embryos and the mutant embryonic stem cells were generated by cultivation in vitro according to the method of U.S. Pat. No. 6,103,523. In FIG. 1, a Southern blot is shown demonstrating the presence of wild-type or knockout alleles of presenilin1 in ES cell lines obtained from blastocysts generated by mating PS1 +/−, PS2 −/− mice.

3. Determination of γ-secretase Activity

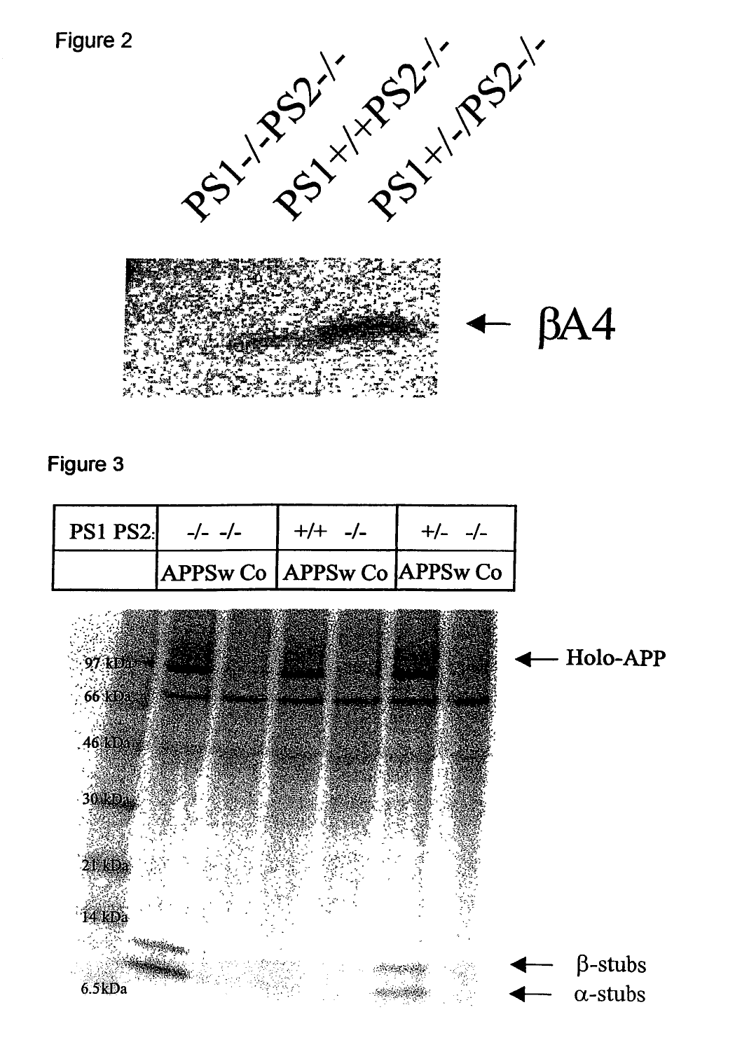

In FIGS. 2 and 3, the analysis of APP processing, containing the Swedish clinical mutation, in a double presenilin knockout background is presented. It is shown that no amyloid peptide production is observed in the double presenilin-deficient ES cells.

The total gamma-secretase activity in cells is measured by assessing the release of the amyloid peptide in the culture medium from cells transfected with cDNA coding for either wt APP or APP containing the Swedish type of mutation. Measurement is done by ELISA, mass spectometry, western blot or double immune precipitation in combination with phosphor imaging or by any other means that allow determination of the amount of amyloid peptide secreted into the medium. The secretion of amyloid peptide in cells expressing wild-type PS1 is taken as reference. Increases or decreases in amyloid peptide secretion by the test cells are expressed as the fraction of the reference.

4. Differentiation of Double Presenilin-deficient ES Cells

Stem cells have both the capacity to self-renew, that is, to divide and create additional stem cells, and also to differentiate along a specified molecular pathway. Embryonic stem cells are very nearly totipotent, reserving the elite privileges of choosing among most if not all of the differentiation pathways that specify the animal. Upon LIF withdrawal, ES cells cultured on non-adhesive tissue culture surface spontaneously aggregate into embryo-like bodies, where they differentiate and spawn many cell lineages, including beating heart muscle cells, blood islands, neurons, pigmented cells, macrophages, epithelia, and fat-producing adipocytes.

In one specific, but not limited, example aggregates of these mutant, cultured mouse ES cells, can be differentiated into neuronal precursor cells and functional postmitotic neurons (Okabe et al., 1996, Mach. Day. 59, 89). This is achieved by taking aggregates of cultured mutant ES cells and propagating them in medium supplemented with insulin, transferrin, seleniumchloride and fibronectin to select for CNS stem cells. These CNS stem cells are proliferated in the presence of mitogen, bFGF. Further differentiation of the stem cells into mature neurons is achieved by withdrawal of bFGF.

This experimental system provides a powerful tool for analyzing the molecular mechanisms controlling the functions of these neurons in vitro.

In another example, these mutant ES cells can be differentiated into adipocytes. This is achieved by culturing the embryoid bodies in medium containing retinoic acid and subsequently plating them in medium supplemented with insulin and triiodothyronine.

This system provides a model for the further characterization of the role of genes, expressed during the adipocyte development program, like Notch-1, which is required for adipogenesis.

5. Development of a Reporter System for Gamma-secretase and Use of the System in Cell Lines

The principle of the assay is depicted in FIG. 4A. An APP/Notch chimeric protein is generated. This protein contains the APP ecto- and transmembrane domains, fused to the Notch intracellular domain. The Notch intracellular domain (NIC), when cleaved, translocates to the nucleus and activates a reporter gene construct containing a defined part of the Hes 1 promotor controlling Luciferase expression. Thus, proteolytic cleavage of the chimeric protein is directly linked to luciferase activity. The chimeric protein and the luciferase reporter are transiently or stably transfected in Hela-cells, HEK293 cells, COS-cells, embryonic stem cells and others. The chimeric protein is cleaved by α-secretase, β-secretase and γ-secretase. The γ-secretase cleavage of the construct is dependent on PS expression. In ES cells lacking PS1 and PS2 transfected with the luciferase reporter and the chimeric protein and a control plasmid, no significant luciferase activity is induced. If an expression plasmid coding for PS1 is cotransfected, however, luciferase activity is induced. Transfection experiments using the intracellular domain of the chimeric protein alone (bypassing the need for γ-cleavage) results in much stronger activity of the luciferase reporter in Hela cells than obtained with the APP/Notch chimeric. Therefore, both decreased and increased γ-secretase cleavage can be assessed with this assay.

This assay can be used as a screening assay for compounds that inhibit or stimulate γ-secretase activity. In such assays compounds are added to cells expressing the chimeric protein and reporter. After a defined period of time (24 or 48 hours, but shorter periods of time can be chosen), luciferase activity is measured in the treated cells and in the control cells. Changes in luciferase activity are an indicator of decreased or increased γ-secretase activity. Compounds that selectively decrease luciferase activity in cells expressing the chimeric protein and reporter but not in cells transfected with the intracellular part of the chimeric protein alone are not toxic to the cells and are likely specific inhibitors for the γ-secretase.

This assay can also be used to screen for cDNA's coding for proteins that modulate γ-secretase activity. In this type of experiment, cDNA's from a cDNA library either using classical transfection protocols or using viral transduction (adeno-, Semliki Forest- or other viral vectors) are transfected into cells or cell lines expressing transiently or stably the chimeric protein and the reporter. Positive hits (significant up or down regulation of luciferase activity) can be selected from these screens. The plasmids coding for individual cDNA's or for pools of cDNAs can be used for further screens in the same assay or for experiments in neurons or cell lines to confirm their effects in APP processing. Pools of plasmids can be further subdivided and tested in consecutive rounds of transfection and subdivision until plasmids encoding a single type of cDNA are obtained. This cDNA is anticipated to encode γ-secretase, an active γ-secretase fragment or modulators that either activate or inhibit this enzyme. Further validation of positive clones can be performed by transfecting or transducing the obtained cDNA's into neuronal cells or in cell lines to analyze APP processing. These cells can be transfected with APP constructs encoding human APP.

A third application of the assay is the screening for ligands of APP. This is based on the observation that binding of ligands to Notch induces proteolytic processing of Notch and cell signaling. It is expected that in analogy, APP ligands binding to the ectodomain of the chimeric protein will induce γ-secretase processing, which consecutively will cause enhanced luciferase activity. Serum and plasma, cerebrospinal fluid, or other body fluids, cell extracts, conditioned medium of cells in culture, membrane-enriched extracts of cells in culture, membrane-enriched fractions of tissues, in particular brain or fractions thereof, can be used as a starting material and added to (stably) transfected chimeric protein-expressing cells. If these materials contain APP ligands, it is expected that they will result in increased luciferase activity.

Further purification of these ligands can be obtained by classical fractionation (gel filtration, anion exchange or other chromatographic procedures, ultracentrifugation or specific precipitation using high salt concentrations and other methods). Purified material can be sequenced using Edman degradation or Maldi-Toff. It is also possible to use cells transfected with cDNA's or pools of cDNA's, the chimeric construct and the reporter and to screen for enhanced luciferase activity. If a membrane bound ligand for APP is expressed in these cells, it is predicted that γ-secretase activity and consequently luciferase activity will be increased. By subdividing the plasmid pools and transfecting these subpools in the cells, and by screening these subpools for their effect in the assay, it is possible to obtain progressively enriched pools of plasmids encoding cDNA's for potential ligands of APP. By repeating this type of experiment until one single cDNA clone is obtained, it is possible to identify APP ligands.

6. Development of a Cell-free Assay

The mutant ES cell line can be used as the basis for a cell-free assay following the generation and release of transport vesicles from endoplasmic reticulum. The same type of studies has been critical for the unraveling of the molecular sorting machinery in eukaryotic cells. One can isolate ER-fractions from wild-type and PS1−/− mice and the export budding from the ER can be reconstituted using small GTP binding proteins and recombinant COPII coat proteins. Newly formed vesicles can be isolated and analyzed for amyloid peptide generation. The molecular composition of the vesicles can be analyzed using 2D gel electrophoresis combined with amino acid sequencing. Cross-linking experiments allow identification of components of the postulated presenilin-APP-γ-secretase complex. By comparing protein profiles from isolated vesicles generated from the double deficient (PS −/−) cell line with that from wild-type, PS expressing ES cells, it is possible to identify proteins that are differentially present in the vesicles. Analyses by two-dimensional PAGE and amino acid sequencing of differentially detected protein spots will yield new proteins whose processing and transport is regulated by presenilins. Furthermore it is envisaged that proteins like γ-secretase or other proteases that are dependent on the presence of presenilins will be either decreased or increased in these samples. Further amino acid sequencing of these spots will yield γ-secretase and other protease candidates involved in RIP (regulated intramembrane proteolysis) (Brown et al., 2000, Cell 100, 391).

7. Inhibitors of β-amyloid42 Peptide Production

In another example, the mutant ES cell line is transformed with the wild-type PS1 gene, resulting in transformant 1, while transformant 2 is the mutant ES cell line genetically transformed with a specific familial Alzheimer's disease causing mutation in PS1 or PS2 or a combination of mutations. It is expected from the state of the art that transformant 2 predominantly produces the amyloid-β-42 form whereas transformant 1 mostly produces the non-pathogenic amyloid-β-40 peptide. These two transformants can be used in a differential drug screening approach to identify compounds which inhibit preferentially the amyloid-β-42 formation and not the amyloid-β-40 peptide generation. In a first screening with transformant 2, compounds are identified which specifically inhibit the formation of the amyloid-β-42 peptide. Specific monoclonal antibodies exist which can differentiate between the amyloid-β-42 and amyloid-β-40 peptides. In a second step, the compounds identified in this first screening are applied on transformant 1. Subsequently, specific compounds are identified which do not have an effect on the amyloid-β-40 secretion. Furthermore, the system can be optimized in a high-throughput way so that large collections of existing chemical compound libraries can be quickly and efficiently validated. The discovery of such desirable compounds has traditionally been carried out either by random screening of molecules (produced through chemical synthesis or isolated from natural sources, for example, see K. Nakanishi, Acta Pharm. Nord, 1992, 4, 319); or by using a so-called “rational” approach involving identification of a lead-structure and optimization of its properties through numerous cycles of structural redesign and biological testing (for example, see Testa, B. & Kier, L. B. Med. Res. Rev. 1991, 11, 35-48 and Rotstein, S. H. & Mureko, M. J. Med. Chem. 1993, 36, 1700). Since most useful drugs have been discovered not through the “rational” approach but through the screening or randomly chosen compounds, a hybrid approach to drug discovery has recently emerged which is based on the use of combinatorial chemistry to construct huge libraries of randomly built chemical structures which are screened for specific biological activities. (Brenner, S. & Lamer, R. A. Proc. Natl. Acad. Sci. USA, 1992, 89, 5381.)

II. Materials and Methods

FIG. 4A the construct and the principle of the assay is schematically represented. DNA constructs (FIG. 4B) were generated as follows:

The fragment of the murine HES-1 promotor containing nucleotides 7 to 251 (Takebayashi K., et al., J Biol Chem. 1994, 269(7), 5150-6) was isolated by polymerase chain reaction (PCR) using the following oligonucleotides: 5′-CTCAGGCGCGCGCCATTG-3′ (SEQ. ID NO: 1) and 5′-AGAGGTAGACAGGGGATTCC -3′ (SEQ. ID NO: 2). As a template, the pGL2HES1luc (Jarriault S., et al., Nature 1995, 377, 355-358) was used.

The amplified fragment was subcloned in a pGEM-T vector (Promega) and verified by sequencing. The coding sequence of the luciferase gene from the pGL2 basic vector (Promega) was subcloned (as a HindIII (blunded)- SaII fragment) downstream the HES-1 promotor (SpeI (blunded)-SaII vector) giving rise to pHES1-luc construct. The PGKneo selection marker cassette from Adra et al. (Gene, 105, 263-267, 1991) was introduced in the SaII site of pHES-luc as a SaII-XhoI fragment, resulting in pHES-1luc-neo, which was to generate stable cell lines.

APP:NIC (FIGS. 4A and 4B) fusion constructs, also called chimeric proteins, were made using the human APP695 sequence and the mouse Notch-1 sequence. The APP encoding sequence (residues −20 bp to +1992 bp, the ATG of the APP open reading frame being number 1, was constructed by joining the SmaI-SacI fragment encoding bases (−20 bp to +1692) to a PCR fragment encoding bases (+1693 bp to +1992 bp) generating a unique EcorV site and a Myc tag (EQKLISEEDL) (SEQ. ID NO: 3) at the 3′ end. The oligonucleotides used for PCR were: 5′-AACCACCGTGGAGCTCCTTC-3′ (SEQ. ID NO: 4) and 5′-CCAAGCTTCTACAAGTCCTCTTCAGAAATCAGCTTTTGCTCGTTAACGATATCGTCAACCTCCACCACACCATG-3′ (SEQ. ID NO: 5).

Three different cDNA fragments encoding part of the Notch-1 intracellular domain (+5286 bp to +6291 bp, +5286 bp to +7251 bp, +5286bp to +7554 bp respectively, bp +1 being the A of the ATG) were generated by PCR on a mouse brain cDNA library and subcloned into the EcoRV site, giving rise to APP:NIC2, APP:NIC, and APP:NIC1 (see FIG. 4B). The oligonucletides used were: 5′-CACCCGGGTTCCCTGAGGGTTTCAAAGT-3′ (SEQ. ID NO: 6), 5′-CCGCACGATATCGTGGTG-3′ (SEQ. ID NO: 7), 5′-GCGTTAACATCTGCCTGACTGGGCTC-3′ (SEQ. ID NO: 8) and 5′-CAGTTAACGGTGGTGGGCGGGCTGGAGAT-3′ (SEQ. ID NO: 9). An SV40 polyadenylation signal (isolated from pSG5, Stratagene) was added as indicated in FIG. 4B. The mouse Pgk-1 promotor (−456 bp to −18 bp, Adra, C. N., Gene 1987, 60, 65-74) or the SV40 early promotor from pSG5 (Stratagene) was cloned in the unique SmaI site of the fusion constructs.

The K595N;M596L (numbering as in APP 695, the M encoded by the start ATG is 1) Swedish mutation was introduced into the pSG5APP:NIC plasmid by site-directed mutagenesis (Stratagene) using the following primer 5′-GGAGATCTCTGAAGTGAATCTGGATGCAGAATTCCGAC-3′ (SEQ. ID NO: 10). This construct was called pSG5APPSw:NIC. PSG5βA4:NIC was generated by replacing the APP ectodomain of pSG5APP:NIC with the APPC99 stub. APPC99 contains the carboxyterminal 99 amino acid residues of the APP sequence (thus starts with the β-cleavage site in APP). To obtain the correct cleavage by signal peptidase in the APPC99 stub, an extra DA motif was added between the signal sequence and the β-cleavage site. An NIC construct containing only the intracellular part of the chimeric protein was generated by PCR with the following oligonucleotides 5′-AGGATCCATGGTGCTGCTGTCCCGCAAGCGCCGGCGGCAGCATGGCCAGCTCTGGTTCCCTGAGGGTTTCAAAGTGT-3′ (SEQ. ID NO: 11) and 5′-GCGTTAACATCTGCCTGACTGGGCTC-3 (SEQ. ID NO: 12). The fusion AFP:NIC and the NIC construct were cloned in pcDNAzeo (Invitrogen) to generate stable cell lines.

The complete cDNA maps of the pHes1-Luc construct and the promotorless APF:NICD are included.

The constructs discussed above were transfected in COS cells, in Hela cells, in ES cells, and in HEK293 cells. We discuss the Hela cells here in more detail as one example.

Hela (provided from the ATCC culture collection) cells were cultured in 12-well plates in DME/F12 medium (Gibco, BRL) supplemented with 10% Fet al. Bovine Serum. Plasmids were transfected using Fugene according to the manufacturer (Roche). The transfection reagent:DNA ratio was 6:1. Five hours before transfection the culture medium was replaced by DME/F12 without serum. Each well was transfected with a total of300 ng DNA consisting of 50 ng pHes1-luc and 250 ng of one of the mNotch plasmids discussed above (plasmids containing NIC) or empty pSG5 vector (control). Luciferase activity reflecting activation of the Hes-1 promotor fragment was measured 48 h after transfection with the luciferase assay system of Promega using a luminometer. All experiments were performed in duplicate or triplicate and repeated at least two times. Luciferase induction factors were determined as the ratio between the mean luciferase activities of the mNotch variants (as indicated) and the mean luciferase activities of the empty pSG5 vector.

Stable transfected HELA cells were obtained by electroporation (500 V/cm, 960 μF) of 3×10⁶ cells in the presence of 10 μg linearized (SaII) pHes1-luc-neo DNA. After 24 h 500 μg/ml G418 was supplemented to the media. Resistant cells were picked, expanded, frozen and analyzed. Cell line designated 105, giving low background luciferase activity and a 10-fold induction after transfection with pSG5APP:NIC, was reelectropored in the presence of 10 μg of linearized (ScaI) pCDNAzeoAPP:NIC or pCDNAzeoNIC DNA. After positive selection in media containing zeocin (20 μg/ml), resistant cells were picked, expanded, frozen and analyzed. Both APP:NIC cell lines designated 13 and 17 are at least stable for 8 passages and result in a 50-fold induction factor. One NIC cell line, designated 3, is stable for at least 4 passages, giving a 25-fold induction of luciferase activity.

What is claimed is:

1. A mutant embryonic stem cell line, wherein the γ-secretase activity is reduced by at least 90% compared to the γ-secretase activity in wild-type embryonic stem cell lines.

2. The mutant embryonic stem cell line of claim 1, wherein said mutant embryonic stem cell line comprises a double mutant embryonic stem cell line of a presenilin 1⁻¹⁻ presenilin 2⁻¹⁻ knock-out mouse origin.

3. The mutant embryonic stem cell line of claim 1 or 2, wherein said γ-secretase activity is reduced by at least 99%.

4. The mutant embryonic stem cell line of claim 1 or 2, wherein said γ-secretase activity is reduced by at least 99.9%.

5. The mutant embryonic stem cell line of any of claims 1-4, wherein said mutant embryonic stem cell line is differentiated.

6. The mutant embryonic stem cell line of any of claims 1-5, wherein said mutant embryonic stem cell line is differentiated into a post-mitotic neuron.

7. The mutant embryonic stem cell line of any of claims 1-5, wherein said mutant embryonic stem cell line is differentiated into an adipocyte.

8. A method of producing the mutant embryonic stem cell line of any of claims 1-7 comprising:
rescuing blastocysts from double presenilin knockout mice; and
cultivating said blastocysts in vitro.

9. A method of identifying a gene coding for a protein having γ-secretase activity comprising:
transfecting the mutant embryonic stem cell line of any of claims 1-7 with at least one gene coding for a protein whose ability to have γ-secretase activity is to be determined; and
monitoring said γ-secretase activity in said mutant embryonic stem cell line.

10. A method of identifying a compound which specifically interferes with the formation of the Aβ42-peptide and not with the formation of the Aβ-40 peptide comprising:
transfecting the mutant embryonic stem cell line of any of claims 1-7 with at least one mutated gene coding for a protein selected from the group consisting of presenilin 1, presenilin 2, and amyloid β precursor protein, to produce a transfected stem cell line;
exposing said transfected stem cell line to at least one compound whose ability to interfere with the formation of the Aβ-42 peptide and not with the formation of the Aβ-40 peptide is to be determined; and
monitoring said formation of Aβ-42 peptide.

11. A process for producing a pharmaceutical composition comprising:
identifying a gene encoding for a protein having γ-secretase activity according to the method of claim 9 or 10; and
mixing the gene or compound identified or a derivative or homologue thereof with a pharmaceutically acceptable carrier.

12. A method of making a transgenic mouse comprising:
transfecting the mutant embryonic cell line of any of claims 1-4 with a pathogenic presenillin Alzheimer disease causing gene;
injecting the resulting transfected mutant embryonic cell line into a blastocyst; and
implanting said injected blastocyst into a female mouse.

13. A reporter system for detecting intramembrane proteolytic processing comprising:
a chimeric molecule comprising a fusion between a transcription factor and a transmembrane domain that is known to be a substrate for proteolytic processing; and
a reporter construct that detects said proteolytic processing of said chimeric transcription factor.

14. The reporter system of claim 13 wherein said chimeric molecule comprises a fusion between the intracellular domain of Notch and the transmembrane domain of APP.

15. The reporter system of claim 13 wherein said chimeric molecule comprises SEQ ID NO: 13.

16. A method of screening for modulators and/or proteases for intramembrane proteolytic processing comprising:
constructing a reporter cell line by transfecting a cell line with the reporter system of any of claims 13-15;
treating said reporter cell line with at least one compound or transfecting said reporter cell line with at least one gene; and
comparing the expression of the reporter gene present in said reporter cell line with the expression of the reporter gene in the non-treated or non-transfected reporter cell line.

17. A process for producing a pharmaceutical composition comprising:
screening for modulators and/or proteases for intramembrane proteolytic processing with the method of any of claims 13-16; and
mixing the modulator and/or protease identified or a derivative or homologue thereof with a pharmaceutically acceptable carrier.

18. A method of constructing the reporter system of any of claims 13-15 comprising:
splicing a genetic element encoding a transcription factor to a genetic element encoding a transmembrane domain that is known to be a substrate for proteolytic processing, to produce the chimeric molecule; and
splicing a genetic element that is responsive to said transcription factor to a reporter gene, to produce the reporter construct.
